(19) United States
(12) Patent Application Publication
(10) Pub. No.: US 2019/0304582 A1
(43) Pub. Date: Oct. 3, 2019

(54) Methods and System for Real Time, Cognitive Integration with Clinical Decision Support Systems featuring Interoperable Data Exchange on Cloud-Based and Blockchain Networks

(71) Applicants: Stephen Blumenthal, Newfield, NY (US); Dale Potter, Round Rock, TX (US); Patient Oncology Portal, Inc., Newfield, NY (US)

(72) Inventors: Stephen Blumenthal, Newfield, NY (US); Dale Potter, Round Rock, TX (US); Kevin Simon, Mahwah, NJ (US); Baxter Garcia, Ridgefield, CT (US)

(21) Appl. No.: 16/374,683

(22) Filed: Apr. 3, 2019

Related U.S. Application Data
(60) Provisional application No. 62/652,187, filed on Apr. 3, 2018.

(51) Int. Cl. G16H 15/00; G06F 21/62; G06F 9/451; G06F 16/245; G16H 10/20; G06N 20/00

(52) U.S. Cl. CPC G16H 15/00; G06F 21/6245; G06F 9/451; G10L 15/26; G16H 10/20; G06N 20/00; G06F 16/245

(57) ABSTRACT

An informatics platform comprising method, system, and computer program is provided for interacting with clinical decision support systems such as the IBM Watson for Oncology, for the purpose of utilizing cognitive processing, machine learning and natural language processing to facilitate the manual to semi-automatic to automatic acquisition of a patient's electronic medical record data points, into a CDS system for populating the attributes questionnaire, for generating a patient diagnostic report of treatment recommendations. The platform supports deployment on several informatics architectures including client-server, cloud-based and blockchain.

In a typical embodiment, the system is deployed as a Software-as-a-Service (SAAS) application; it implements a cloud-based, real-time architecture comprising: (a) an adaptive user interface providing responsive dashboards for real-time data presentation and user interaction, (b) a hub controller for real-time data flow transformation, (c) a data validation engine which incorporates cognitive natural language processing (NLP) to extract structured and unstructured patient record data from the EHR and employs methods that provide an optimally automated input to the CDS, (d) cloud storage of aggregated CDS data and other clinical datasets, and (e) plug-in support for additional cognitive capabilities such as predictive analytics and data mining.

### CROSS REFERENCE TO RELATED APPLICATIONS

This application claims priority from U.S. Provisional Application No. 62/652,187 filed on Apr. 3, 2018, the disclosure of which is incorporated herein by reference in its entirety.

### FIELD OF INVENTION

The present invention relates to clinical decision support systems (CDS) and to medical informatics platforms that support CDS systems, in particular those utilizing artificial intelligence. The invention design is an embodiment of a ‘Real Time Health System’, i.e. intelligent and aware, connected and collaborative, realtime and adaptive, integrated and interoperable, and mobile and IoT-capable. The inventive platform is compatible with different health IT infrastructures including client-server and cloud-based. The inventive design is particularly well adapted to the blockchain infrastructure.

### BACKGROUND OF THE INVENTION

Historically, medical record systems were paper-based, requiring massive amounts of documentation to track the myriad daily transactions involved in hospital operations. The development of the Electronic Health Record (EHR) began in the mid-60's and the first computerized patient record system went live in 1973. Subsequently, hospitals electronically automated their operations with a mixed variety of off-the-shelf commercial software and home-grown customized solutions. These electronic solutions were often incompatible from one internal department (e.g., accounting, pharmacy, lab) to another, and rarely interoperable with a different hospital system.

As the desire for a unified hospital EHR system grew, a number of companies entered the field with comprehensive software platforms that integrated with all aspects of the hospital operations. Some of the key players that emerged to dominate the EHR industry in the US were Cerner, Epic, McKesson, AllScripts and MediTech. Despite industry consolidation and consensus among experts in all sectors of healthcare on the need for common standards and EHR interoperability, achieving this goal has been elusive. Plagued by issues related to legacy systems, the EHR vendors have had to perform numerous customizations on each hospital deployment, thereby defeating the prospects of interoperability. The Obama Administration, through the HITECH Act of 2008, provided incentive funding for hospitals to upgrade and modernize their EHR systems but these efforts have still fallen short; as a result integration of 3d party CDS systems such as IBM Watson for Oncology into hospital workflows remains a challenging task.

With CDS platforms, a key issue centers on the patient electronic medical record (EMR) data and the requirement to aggregate this data as input to the CDS. Ideally, the patient's EHR chart would contain the totality of required data for performing CDS analysis. In practice, though, there may often be missing or incorrect data, requiring laborious manual searches and correction procedures in order to properly update the patient chart.

In an effort to address workflow limitations such as that described above, and aggressively move towards achieving EHR interoperability, the major EHR vendors have in recent years agreed to support a set of open source API (Application Programming Interface) standards.

As previously noted, adoption of EMRs and EHRs has proceeded apace, while data interoperability among health systems remains a major challenge. Incompatible EHR systems prevent seamless data exchange while, within EHRs, a lack of industry-accepted standards has led to a proliferation of siloed clinical applications with weak or non-existent data sharing capacities.

More recently, new standards have emerged with the potential to radically alter the landscape of health data interoperability; these include HL7 FHIR (Fast HealthCare Interoperability Resources), SMART (Substitutable Medical Applications, Reusable Technologies), CCDA (Consolidated Clinical Document Architecture), OAuth2 and others. Many of the leading EHR vendors have signed on to fully support these new open standards.

Clinical decision support systems (CDS) are an increasingly significant tool for health providers. A clinical decision support system (CDS) is a health information technology that is designed to provide physicians and other health professionals with clinical decision support at the point of care, i.e., assistance with clinical decision-making tasks. CDS technologies cover a wide spectrum, but can be loosely categorized as ‘rules-based’, which are typically older systems, and artificial intelligence (AI) based systems, which characterize the current generation.

Those particular systems utilizing artificial intelligence technologies such as natural language processing and machine learning are quickly establishing themselves as valuable, even indispensable components in modern healthcare; they are demonstrating value in multiple areas including data mining of medical records, tailoring personalized diagnosis, designing treatment plans, assisting with repetitive jobs, precision medicine and drug discovery.

Within this category the clear leader is IBM Watson Health with R&D investments in the billions of dollars, a network of top US hospitals as beta test sites and numerous hospital deployments already underway in regions such as Asia-Pacific and Latin America. Smaller competitors are also active and expanding their market footprint including Flatiron, NantHealth/Evita, and McKesson.

For EHR providers the lack of integration and interoperability within and among EHR systems remains a major factor leading to workflow inefficiencies and excessive costs plaguing the US healthcare industry. Cognitive technologies epitomized by IBM Watson Health hold out the promise of mitigating these issues and streamlining clinical workflows in the years ahead.

It is increasingly apparent that a series of trends that began with the digital revolution and the advent of EHRs, are pointing towards a major transformation within the healthcare industry. The influential Gartner Group has analyzed these trends and identified the real-time health system (RTHS) as the next-generation evolution in healthcare. Gartner states that this transformation will occur over the next 5-10 years, driven by “healthcare reform, a technology-enabled mobile workforce, the inexorable progression of medical knowledge, changing demand models, and an abiding need to improve care quality and the patient experience. To succeed in this new environment, healthcare providers will need to embrace the RTHS as their next-generation operations and technology archetype.”

### SUMMARY OF THE INVENTION

The present invention is designed to enable the transition towards real-time health systems through an innovative informatics platform designed to support and enhance operations for intelligent Clinical Decision Support Systems (CDS) such as IBM's Watson For Oncology (WfO). The platform implements a real-time system architecture while leveraging cognitive computing technologies including NLP and machine learning to provide increased automation for CDS workflows, boost user engagement, and promote integration and interoperability across disparate hospital EHR systems.

The use of cognitive, AI technologies ensures that the inventive platform ‘learns’ over time, continually improving its operations. AI will be used to optimize the performance of the core system modules and can also be applied to the operations of the system globally, enabling system administrators to assess performance on an ongoing basis.

The real-time capabilities of the inventive platform are explicitly designed to optimize performance in the approaching era of Internet-of-Things (IoT) devices such as biometric sensors and health-app enabled smart-watches and phones that will be increasingly important in the patients' health experience.

The portal platform is equally capable of being implemented on several alternate informatics architectures:

Client-Server
The major EHR vendors today, e.g. Cerner, Epic, McKesson, are primarily client-server based although there is intense pressure to migrate to cloud-based systems. Legacy client-server installations feature in-house servers that deliver application software to end-user clients who may be using, e.g. PCs or mobile devices. Client-server has the advantage of offering a high level of control for maintenance, security, etc. to the host institution; however, a downside is the complexity and expense of having to maintain in-house servers or datacenters spanning a proprietary network.

Cloud-Based
Cloud-based architecture has faced skepticism in terms of healthcare data security but offers major advantages in terms of ease of software deployment and maintenance and corresponding reduced costs. The advantages of shifting responsibilities for these tasks to the cloud vendor can offset the disadvantage of diminished control over, eg. network performance, timing of updates, etc.

Blockchain
Blockchain offers a new and different architectural approach which appears poised to disrupt the healthcare industry profoundly in the near future. While cloud technology is on the verge of supplanting legacy client-server systems, cloud-based health systems still have severe limitations in terms of data security and interoperability between healthcare providers, payers and patients. In contrast to both client-server and cloud technologies, blockchain uses a decentralized ‘distributed ledger’ technology that is spread across a network of computers with strong encryption at its core. Blockchain technology offers particular advantages for the portal system described herein with regard to clinical data security and the speed and efficiency of clinical workflows, critical factors in promoting transition to real-time health systems.
To illustrate, a unique digital signature ID can be attached to the blockchain which can then be used to build comprehensive longitudinal or historical patient profile EMR data along with e.g. social ecosystem and behavioral data and data input from biometric sensors. In addition to the security provided by the blockchain data structure, transactions on the blockchain, e.g. updates to the EMR, are registered in near real-time and instantly accessible to all permissioned parties involved with the patient's care.

The inventive platform in one preferred embodiment will be deployed as a SAAS (Software-as-a-Service) application; it implements a cloud-based, real-time architecture comprising: (a) an adaptive user interface providing responsive dashboards for real-time data presentation and user interaction, (b) a hub controller for real-time data flow transformation, (c) a data validation engine which incorporates cognitive natural language processing (NLP) to extract structured and unstructured patient record data from the EHR and employs methods that provide an optimally automated input to the CDS, (d) cloud storage of aggregated CDS data and other clinical datasets, (e) plug-in support for additional cognitive capabilities such as predictive analytics and data mining, (f) connectors built on open-standard API's to facilitate connection to third-party apps, (g) API's encapsulating portal functionality, using open-standards such as HL7 FHIR, to facilitate third-party vendors in building platform-compatible apps.

The system is designed for scalability and extensibility to allow customization to accommodate the needs of individual healthcare organizations. Examples of such customization include: customer A needs a solution that automatically links the output of the CDS to the input of the Predictive Analytics (PA). A module with a pre-stored set of queries to be automatically run on the PA system for each of a particular set of patients, customer B wishes to automatically aggregate clinical data from certain patients as part of a planned research study, customer C plans to deploy biometric monitoring for select patients and is interested in setting the CDS diagnostic report for auto-run to generate reports on these patients when certain events are registered in real time from the sensors.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

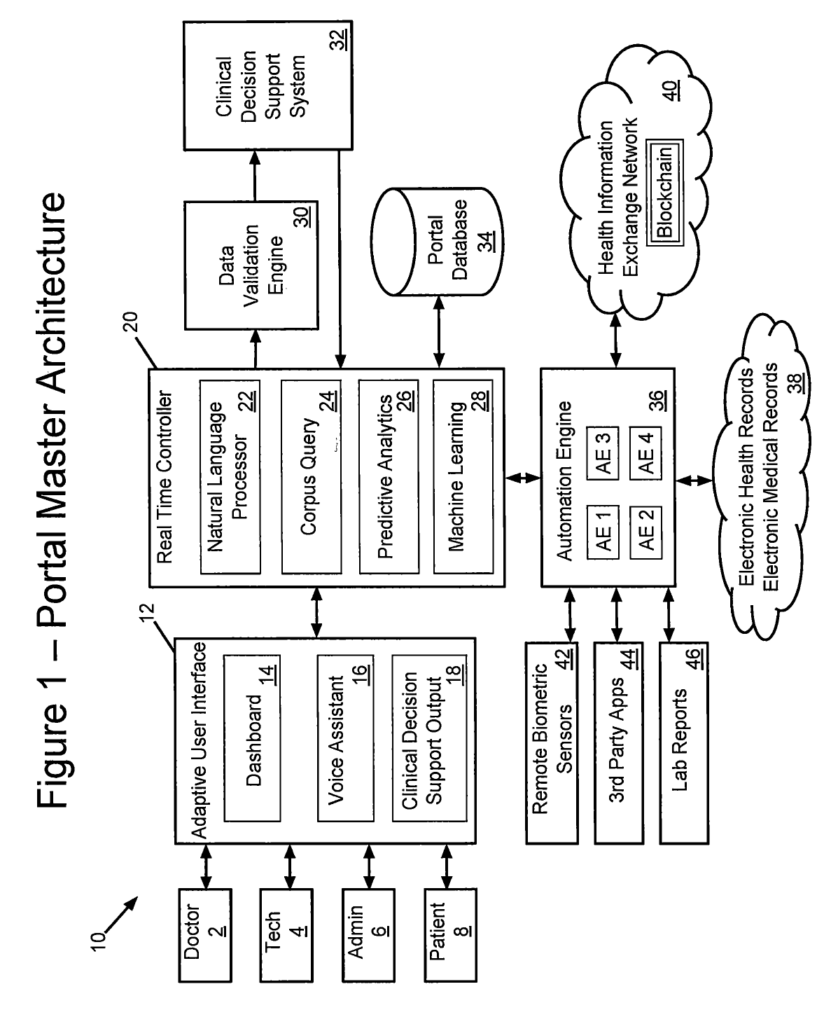

### FIG. 1 Master Architecture

FIG. 1 diagrammatically depicts Portal Master Architecture comprised of an Adaptive User Interface (AUI), a Real Time Controller (RTC), a Data Validation Engine (DVE), an Automation Engine (AutoE) and a Clinical Decision Support System, (CDS), Portal Database and connectors to host and external entities.

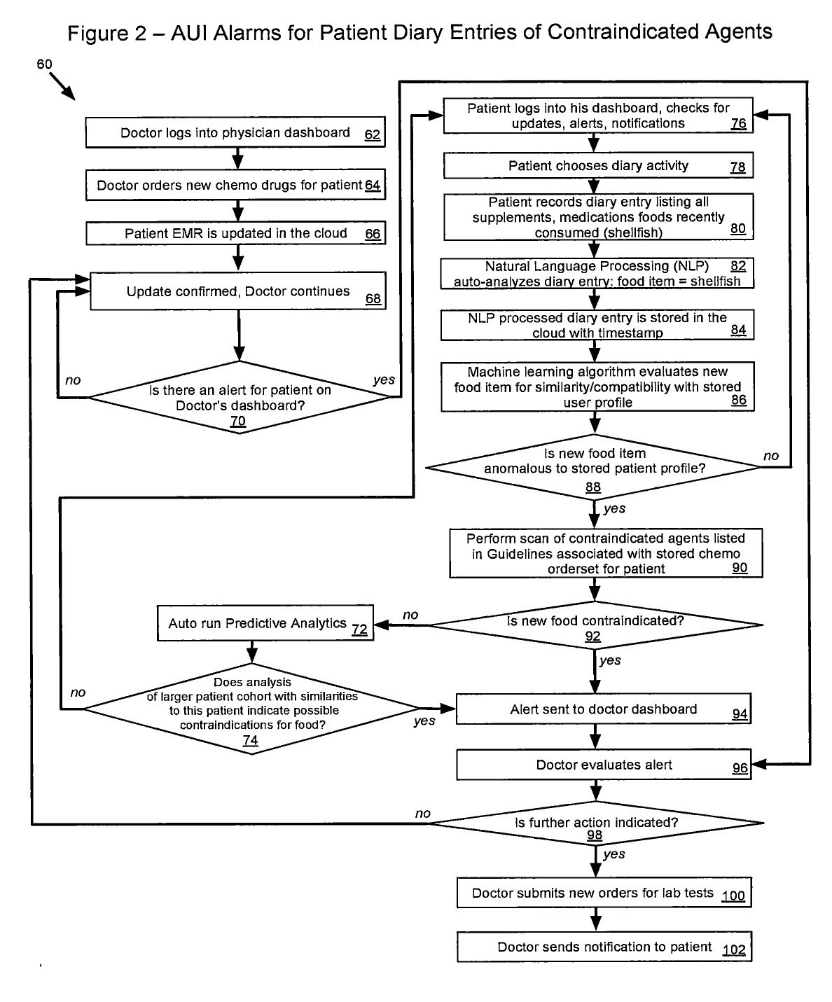

FIG. 2 AUI Alarms from Patient Diary Entries

FIG. 2 demonstrates an embodiment of the inventive real-time, intelligent operations of the portal system in generating physician alerts stemming from Patient Diary entries of a wide range of patient centric issues, such as; of possible contraindicated agents consumed by the patient, diet and radical changes in metabolic indicators

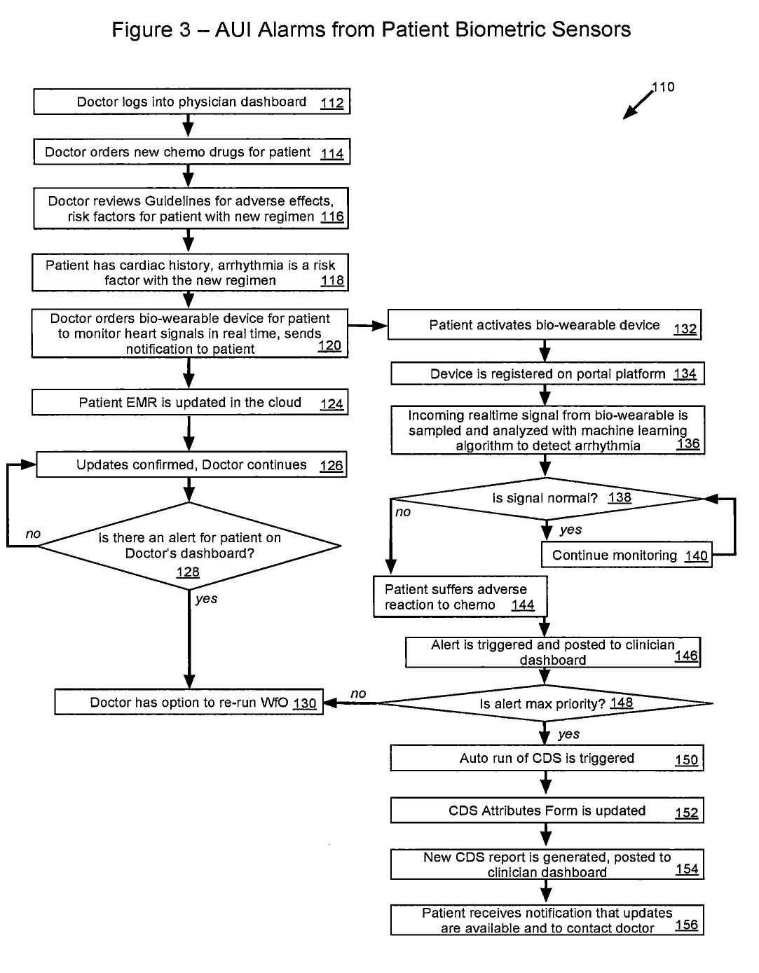

FIG. 3 AUI Alarms from Patient BioMetric Sensors

The embodiment diagrammed in FIG. 3 demonstrates an embodiment of the inventive real-time, intelligent operations of the portal system in generating physician alerts based on monitoring of signals generated by sensors that may be incorporated in patient bio-wearable devices, such as cardiac monitors.

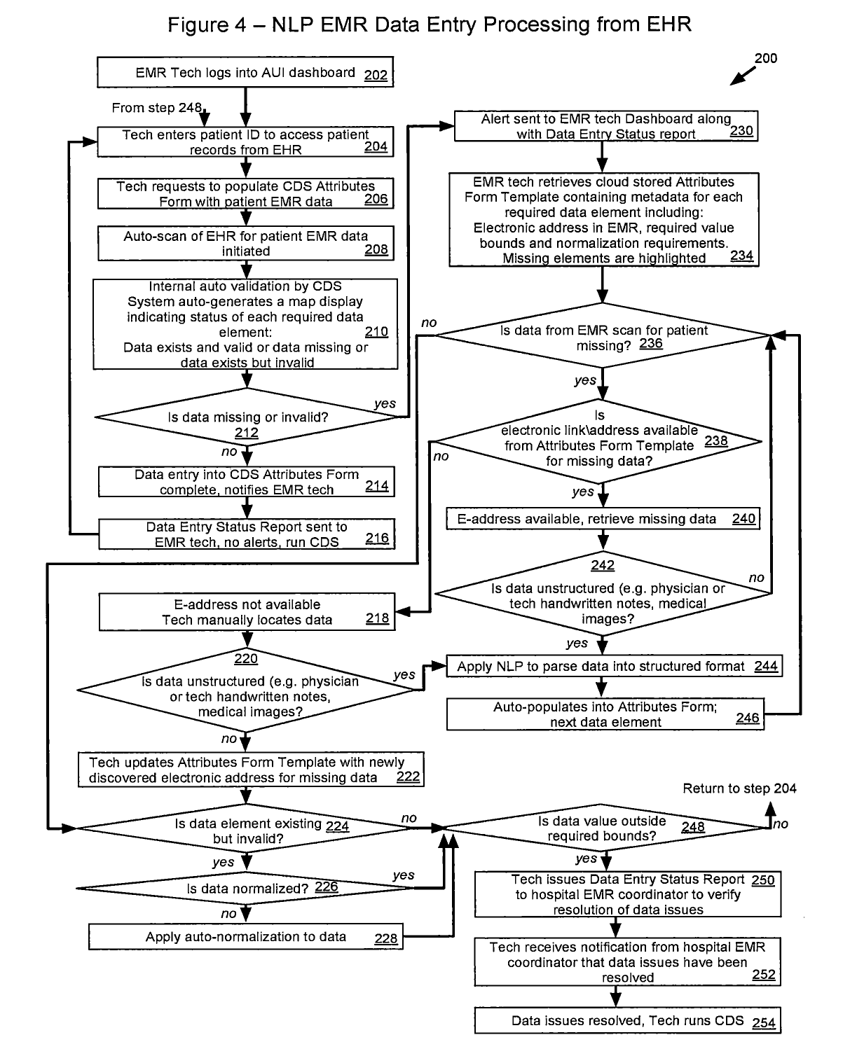

FIG. 4 NLP EMR Data Entry from EHR

The embodiment diagrammed in FIG. 4 demonstrates an embodiment of the inventive real-time, intelligent portal in ingesting data from the EMR which resides in the EHR into the Attributes Form (AF) which is required data entry input for the CDS. This inventive process is designed to transition the workflow from manual to semi-automated and ultimately to fully automated performance.

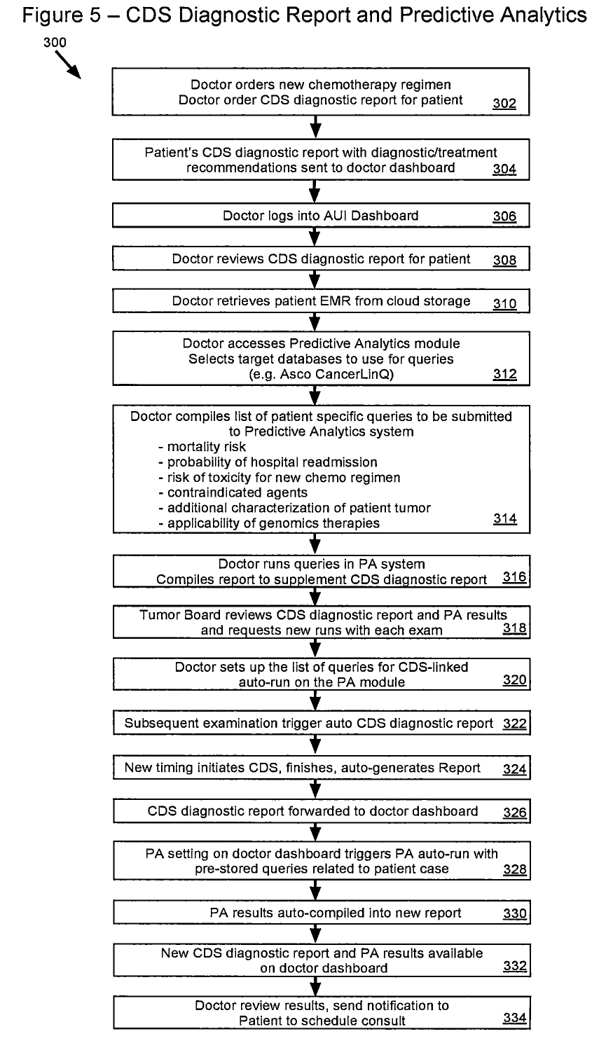

### FIG. 5 CDS and Predictive Analytics

The embodiment diagrammed in FIG. 5 demonstrates an embodiment of the inventive real-time, intelligent operations of the portal system in linking the Predictive Analytics module to the CDS, for the purpose of extracting additional clinical insights to advance the standard of care in the healthcare organization.

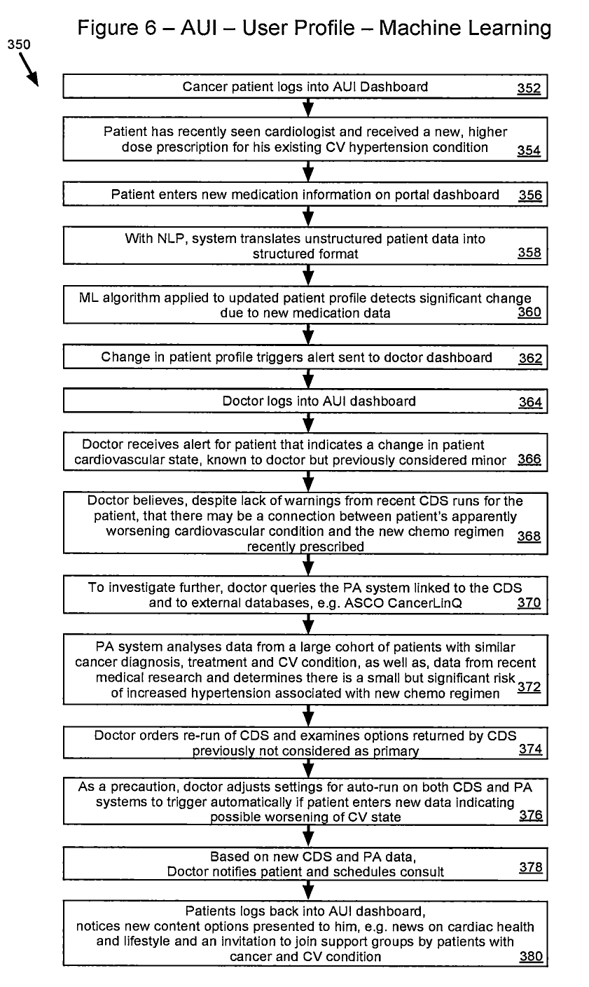

FIG. 6 AUI—User Profile with Machine Learning

The embodiment diagrammed in FIG. 6 demonstrates an embodiment of the inventive real-time, intelligent operations of the portal system Adaptive User Interface which uses machine learning in optimization of the user profile to enhance user engagement.

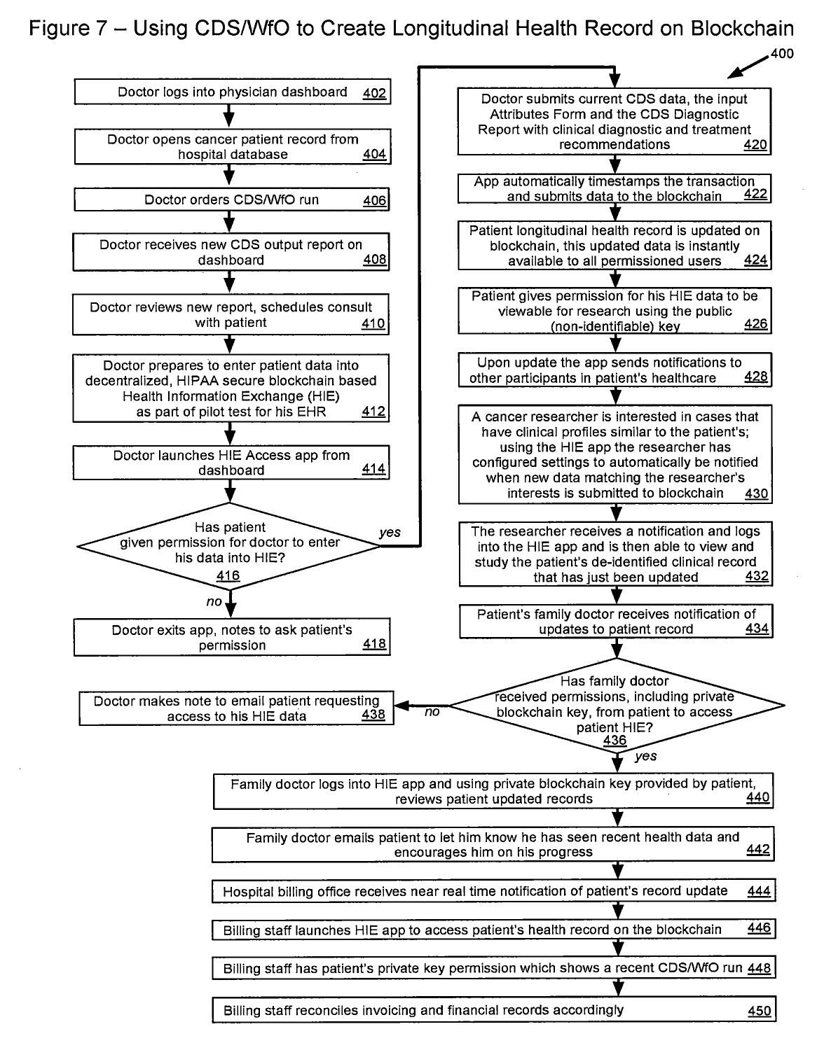

### FIG. 7 Use of CDS to Create Longitudinal Health Record on Blockchain

The embodiment diagrammed in FIG. 7 demonstrates an embodiment of the inventive real-time, intelligent operations of the portal system to create a longitudinal health record on blockchain.

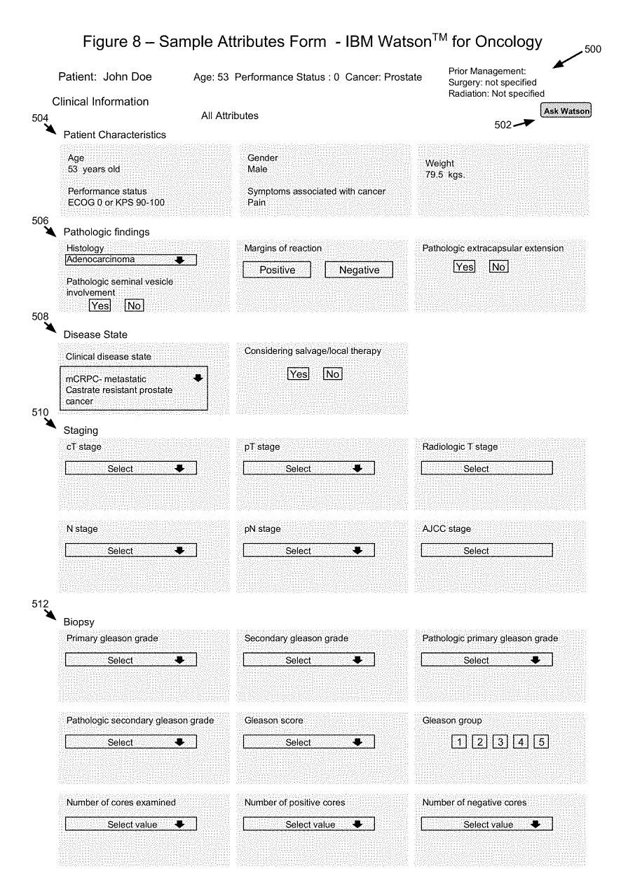

### FIG. 8 Use of CDS Search Template for EMR Insertion of Patient Data

The embodiment diagrammed in FIG. 8 depicts the IBM Watson For Oncology CDS “Attributes” template for inputting the patient's EMR data.

### DETAILED DESCRIPTION OF THE PRESENTLY PREFERRED EMBODIMENTS

1. System Architecture

The inventive portal 10 is diagrammatically depicted in FIG. 1, comprising an Adaptive User Interface (AUI) 12, a Real Time Controller (RTC) 20, a Data Validation Engine (DVE) 30, a Clinical Decision Support System (CDS) 32, a Portal Database 34, and an Automation Engine (AutoE) 36, along with connectors to host (i.e. EHR 38) and external entities (e.g. Health Information Exchange Network 40, Remote Biometric Sensors 42, Third-Party Apps 44 and Lab Reports 46). The AUI 12 includes a dashboard 14, voice assistant 16, and clinical decision output module 18. The RTC 20 includes a Natural Language Processor (NLP) 22, a Corpus Query module 24, a Predictive Analytics module 26, and a Machine Learning module 28. The Automation Engine 36 is comprised of AE 1, AE 2, AE3 and AE 4 for patient EMR data point capture and is connected to remote biometric sensors 42, third party applications 44, aggregating hospital data and laboratory reports interface 46.

An important feature of the inventive portal 10 is its user-centric aspects, which are enabled through state-of-the-art artificial intelligence tools and an architecture optimized for real-time operations. The system compiles user profiles, stored in the portal database 34, which evolve over time, learning more about the user as time goes on and thereby enabling personalization of the system to better fulfill the user needs.

The architecture as shown in FIG. 1 may be implemented as a web application running off web servers and accessible over public or private networks, e.g. the public internet or private secured VPN's. In another embodiment the architecture may be implemented as an app embedded within and hosted by a third-party framework such as an EHR, e.g. the Cerner or Epic EHR systems.

Currently CDS systems within EHRs are principally designed to operate in an on-demand mode separate from or in parallel with other workflows related to the patient care process. This mode of operation is rapidly approaching obsolescence in the emerging healthcare environment where there is an increasing volume of critical real time data as well as real time engagement of the patient and family with their healthcare journey.

A related trend, which has been termed ‘intelligent clinical decision automation’ refers to healthcare processes wherein diagnostic tests are automatically ordered, medications prescribed, referrals scheduled, payer authorizations instantly approved—based on information provided in advance, collected electronically and analyzed intelligently.

The inventive platform 10 has been designed with a Real time Controller 20 and Automation Engine 36 to support the logical requirements for flexible real time operation. The Automation Engine is fully programmable for automating the operations of the CDS, for example, by making it possible to set scheduling parameters (e.g. start time, end time, time between runs) for running the CDS 32 patient diagnostic report (PDR).

One area where real time data occurs involves biometric sensors 42 and other wearable devices such as mobile smartphones and smart watches with heart rate or blood pressure monitoring that generate continuous (unstructured) data. The inventive platform 10 has the capability to capture this type of data, process it using the RTC 20, AutoE 36 and DVE 30 and incorporate the processed real time data into the input Attributes Form for the re-run of the CDS 32, patient diagnostic report.

A. Adaptive User Interface (AUI) 12

The adaptive user interface (AUI) 12 is the user-facing component of the invention, and consists of visual elements, navigation components and content. The AUI 12 may be implemented on a multiplicity of devices, e.g. mobile devices, a smart monitor, or a work station.

The AUI 12 supports a broad range of additional capabilities tailored to the user role, e.g. physician, patient, EMR tech and personalized to the individual user, e.g.: user diary, physician's log, alerts, alarms and notifications, social media links, community announcements, nutritional guidance, current guidelines for treatment protocols, safeguards and side-effects.

The AUI 12 is designed to support a broad array of functions to support users in multiple roles, e.g. physician and patient users, including the following:

1. Dashboard 14

The Dashboard 14 constitutes the UI (user interface)/UX (user experience) presented for the user when they log into the portal system. The dashboard 14 is designed to present curated, real time content and navigation personalized for the authenticated logged-in user. The content and navigation options available on the dashboard 14 are customized to user role, e.g. physician, nurse, lab tech, administrator, patient and personalized for the individual user.

2. Voice Assistant 16

The Voice Assistant 16 (VA) allows the user to communicate with the system through voice. The VA 16 employs an intelligent speech-to-text converter using NLP algorithms available in the NLP module 22 of the RTC 20. Alternatively, a third-party plug-in module can be used for this function.

The user-centric functions described here may be considered similar to other AI-based systems that have recently become commonplace in the consumer market, such as the well-known voice assistants Siri™ from Apple® and Alexa™ from Amazon©, or the recommendation engines used in Netflix®, Amazon and other companies. A voice interface can readily be implemented to operate through the AUI 12 in the inventive portal 10. Natural Language Processing (NLP) 20 performs efficient recognition of the speech converted text by converting the speech converted text into structured data formats.

3. Clinical Decision Support Output 18

Results in the form of patient diagnostic reports (PDR) from the CDS 32 appear on the Dashboard 14. In the example of the WfO CDS 32, this PDR format presents an evidence-based, hierarchically ordered list of diagnostic and treatment options. Physicians readily access the evidence for each presented option by clicking on links to the medical literature, clinical trials data and patient records that were used by the AI system in producing that specific recommendation.

The CDS 32 is an interactive system within the portal architecture 10; accordingly the physician may choose to order a new run of the CDS 32 based on new information for a specific patient. CDS Output 18 is automatically time stamped and archived in the portal database 34 and new reports automatically appear on the dashboard 14.

A novel aspect of the AUI lies in its adaptive capability, which is dynamically linked to the evolving, machine-learning optimized patient profile. The comprehensive profile may be created from a diverse array of datasets including patient demographics, provider orders, diagnoses, procedures, medications, lab values, vital signs, and flow sheet data. While the bulk of data comprising the profile may come initially from the EHR 38, ultimately the expanded profile will include social, behavioral and financial (e.g. claims) data to provide a complete 360 degree view of the patient.

In compiling the profile, NLP 22 of the RTC 20 can be used to parse unstructured data such as physician's notes into a structured format prior to incorporation into the profile. The profile evolves over time providing a longitudinal patient record (LPR) for storage in portal database 34.

The Machine Learning module 18 (ML) may utilize a variety of ML algorithms, such as, e.g. similarity metrics or collaborative filtering, that can be applied to the LPR in order to provide the user through the AUI 12 and dashboard 14 optimized and customized display content and navigational elements that is best adapted to her needs. While mathematically diverse, these ML algorithms 28 can be broadly described as classification, or pattern matching, systems that identify the features specific to that patient and enable comparison with similar patients. Through the AUI 12, the results of the ML 28 analysis is information and content specific to the user which can be provided in the patient context, for example, in the form of news articles, dietary recommendations, listings of current events and support groups customized for a patient's particular diagnosis or treatment regimen.

Users interacting with EHR software are often limited by the inability of these systems to provide an individualized experience that is updated to the present status of the patient and responsive to the full range of their needs for information and communication. While these software systems, such as EHR patient portals, allow the patient a secure login and a HIPAA-compliant browser session, the software design is generally a homogeneous, uniform approach which provides the same navigational structure, visual elements and oftentimes much of the same content to all logged-in users. The ML-based 28 adaptive system as described represents an improvement over the existing approach in providing a personalized and engaging experience for the user.

The AUI 12 as thus described is a further aspect of the platform invention which, in one significant aspect, allows results from CDS analysis 32 to be translated into modifications within the user interface (UI)/user experience (UX), including the components of navigation, visual interface elements and content. For example, a cancer patient receiving chemotherapy for a particular cancer may receive access to a set of health and lifestyle news articles through the AUI 12. Another patient with the same cancer but receiving radiation therapy may receive a different set of information. And as new information is continuously added to the CDS ‘corpus’, i.e., the relevant informational content provided to each individual patient through the AUI 12, can be kept up-to-date, tracking their health journey.

The AUI 12 enables a highly personalized and responsive UI/UX for the individual user which is integrated with other platform technical components such as RTC 20 and the data validation engine 30 along with the capability for real time operation. Further, the platform 10 will provide an optional capability to collect anonymized usage data; cognitive tools such as those provided by the IBM Watson API can be used to analyze the data for usage patterns and leverage insights for further improvement in efficiency and usability of the AUI.

B. Real Time Controller (RTC) 20

The real time controller module 20 functions as the central logic hub of the portal application, managing all transactions and information flow exchanged between the frontend AUI and backend server or cloud modules such as the CDS module 32, as well as connections to portal database storage 34, cloud aggregation of patient data 40, third-party apps 44 and remote biometric sensor 42 interactions. The controller architecture design supports a spectrum of application interaction modalities from on-demand to real time. The controller design also incorporates the use of AI tools 20, 22, 24, 26, 28 in various embodiments.

The RTC 20 supports a range of modular functions and features a scalable and extensible design to allow additional functions to be added as required for particular applications:

1. Natural Language Processing (NLP) 22

NLP 22 employs computer algorithms to translate unstructured data such as handwritten clinician's notes or digital medical images or voice-converted text into a structured data format; once translated, the data can be stored in a database such as Portal Database 34 or analyzed by system modules including the CDS 32, Corpus Query 24, Predictive Analytics 26 and Machine Learning 28 modules.

2. Corpus Query 24

Clinical Decision Support systems such as Watson for Oncology (WfO) are configured to access a large archive of continuously updated data such as patient case records, clinical trials data and medical literature. The Corpus Query 24 module is accessible through the Dashboard 14 to privileged users and enables searching through this internal database.

3. Predictive Analytics 26

Predictive Analytics 26 is an AI module wherein current and historical data is analyzed to make predictions about future occurrences, e.g. whether a patient will suffer an adverse reaction to a drug or require a hospital readmission. The PA module 26 may interoperate with the CDS module 32; it can also operate in a standalone mode in RTC 20. The PA module 26 enables users to evaluate different hypothetical scenarios based on selected target clinical databases.

4. Machine Learning 28

Machine Learning module 28 is an AI module that comprises algorithms capable of ‘learning’ from existing data and discovering hidden patterns in new data. ML 28 is used in a number of contexts in the present system including classifying and optimizing user profiles, identifying anomalies in real time input signals from remote biometric sensors 42 and assessing the operational efficiency of the portal system 10.

Machine Learning module 28 can operate in real time in analyzing incoming signals such as those generated by biometric sensors 42. One example is wearable cardiac monitors which may be worn by patients who have, or are at risk for, cardiovascular disease. In one example, physiological data (Electrocardiogram, Blood Pressure, Galvanic Skin Response) can be combined with clinical data (Age, BMI, Medication and Family History) to be input to an ML algorithm such as a Support Vector Machine (SVM). This type of algorithm is efficient as a binary classifier as in determining whether the patient is ‘At Risk’ or ‘Not at Risk’. The result of this processing may be to generate an instant alert to clinical staff through the AUI 12.

The clinical value of real time platform operations such as those related to biometric sensors is demonstrated in an example where a patient may suffer a major adverse reaction to her therapeutic regimen, e.g. a specific chemotherapy. The sensor data, uploaded to the platform 10 in real time, triggers an instant alert to clinical staff posted to the AUI 12 dashboard 14 and prompting immediate action. As part of the clinical response, the attending clinicians can enter the patient's changed status into the CDS 32 attributes input form and re-run a patient diagnostic report (PDR) which can be compared with previous reports for up-to-the-minute analysis.

A further aspect of real time platform operation can involve enabling clinical staff to run CDS 32 analyses at regularly scheduled intervals. Some CDS 32 systems such as Watson for Oncology (WFO) continuously add to their corpus by ingesting new data, e.g. information on new clinical trials or the most recently published medical literature. Such updated clinical data may alter the treatment and diagnostic recommendations produced by a Watson analysis run and may be significant to a patient's care plan. In this instance, as with the earlier example above, triggered alerts can be used to notify clinical staff—and the patient—of new information.

A further embodiment of the novel benefits of automated CDS 32 operations, which pertains particularly to intelligent CDS systems such as WfO, involves programmed searches and monitoring of research literature or clinical trials data. Systems like IBM WFO are driven by vast databases, or ‘corpus’ containing clinical literature, clinical trials information and other data which are continuously updated. The Corpus Query 24 module makes it possible to program searches of this corpus for information of interest, to particular patient cases. This information can be returned as a recurring report and posted to the physician dashboard 14. In a further embodiment of this methodology, NLP 22 processing will enable the ‘corpus search’ report to be intelligently read for data that may be critical to the patient, enabling alerts to be posted if indicated through cross-checks against the stored patient information.

Note that in the examples above, the portal 10 architecture enables a workflow whereby the CDS 32 can be automatically triggered to run with updated attributes data input and new diagnostic and treatment recommendation results posted to the physician dashboard 14, along with an automatic time stamped notification

The inventive portal architecture 10 has been designed as an extensible, modular and scalable platform, with open standard software protocols such as FHIR facilitating addition of new components to extend functionality. One such ‘pluggable’ component is the Predictive Analytics (PA) module 26. PA is an AI tool designed for the era of Big Data, which enables the extraction of analytics insights from large datasets. In clinical applications, PA 26 has proven itself to be valuable in predicting important variables for the individual patient, such as clinical outcomes, risk for hospital readmission or risk for adverse reaction to treatment, by analysis of the patient data against a large historical cohort of similar patients in the database.

PA 26 can operate against both internal and external databases, e.g. the internal ‘corpus’ database that is part of the CDS 32 WfO system or numerous external clinical databases that are also available, such as those from American Society of Clinical Oncology (ASCO) or National Institute of Health (NIH).

The inventive system also anticipates a linked type of operation wherein the output of the CDS 32 (i.e. a set of recommended diagnoses and treatments) for the patient can serve as input to the PA module which would then run queries to generate predictive data to determine probabilities in terms of e.g, clinical outcome, hospital readmission or length of stay.

C. Automation Engine (AutoE) 36

As shown in FIG. 1, the Automation Engine (AutoE) 36 is comprised of a number of operational functionalities identified as AE 1, AE 2, AE 3 & AE 4. In a further embodiment the AutoE 36 is a secondary logic controller specialized for programmable timing operations. The AutoE 36 may be utilized for a variety of operations including; flexible scheduling and facilitating semi-automated operations for key Portal functions such as data extraction from the EHR into cloud storage, or for scanning EMR patient records for quality control, mapping, tracking, format verifications. This includes correcting, standardizing and optimizing the doctor's unstructured notes for NLP data point capture for improved Attribute capture of patient data fields.

The AutoE 36 substantiates in object oriented programming, multiple customizable instances, for example; all cars have the same basic components, but each one is unique within the context of its specific purpose and use. The AutoE 36 addresses a variety of customizations of various functions relative to the specific use and targeted task, which may be time based or as a dependent operation. There are four different instances of the AutoE 36 which can run separately or in parallel, they are; AE 1 whose primary operation is in the extraction of data from the EMR to facilitate the NLP capture of text fields. The AE 2 whose primary operation is the translation of the text fields captured by the NLP to optimize the population of the Attributes fields for the CDS run. The AE 3 whose primary operation is the predictive ability to learn from previous NLP data capture runs and therefor anticipate which fields will be required by the Attributes fields and deliver them in advance. The AE 4 whose primary operation is in the aggregation of data from a variety of sources, multiple hospitals, biometric devices, outside lab tests, 3^{rd} party cloud relevant data and in providing it to the appropriate stage or operation of the Portal.

In a key embodiment the AE 1 pathway provides for the regulation of interaction between the RTC, the DVE and the EMR controlling the Attributes population of the CDS with its input from the DVE and output back to the primary Real Time Controller, and a secondary path for mapping, tracking and recording the manual to automatic methodology of the EMR data entry.

The extractor operation is key to the first step of NLP getting the data, and provides 2 critical functions 1) the AE 1 handles getting and verifying the patient permissions needed to access the EMR data, accesses and verifies software permission, accesses patient HIPPA permissions, and hospital authorizations needed for EMR removal of security barriers. Obtaining permission for system access will be mandatory.

2) The primary operation is in accessing and pulling the structured and unstructured data fields that then can be put into a text format, based on the EHR systems template report written in the Cerner, Epic, Allscripts EHR system platform, or for any EHR system in use. This is a custom text file report designed to facilitate patient Attributes capture by maximizing the utility of the NLP.

In support of this primary operation is a further embodiment of this AE 1 functionality, in that it extracts data from the EMR. The extractor is an application which pulls patient data both structured and unstructured from a target system such as an EMR. (Cerner or Epic or others) It generates a text file which enables WfO's Natural Language Processing (NLP) to infer the values of required attributes for a patient's particular cancer. Patient data may reside in doctor's notes, lab reports, pathology reports, radiology reports or specific structured fields.

In the case of structured fields, a clinical expert may be required to supervise the correct mapping between source and output values. Not only should the required data appear in the output file(s), but the generated file(s) should be organized in such a way as to maximize the NLP's ability to infer the data.

Depending on the target system, data acquisition may be accomplished via database queries, API calls or both. Each target system, i.e. Epic or Cerner, etc, will require its own unique Extractor. For example the Epic EHR runs ETL (Extract Transform and Load), and as Epic know how their proprietary data base is set up, they read it all, and convert it to a standardized data base SQL. SQL is the Epic standard format which once converted and saved, it then has accessibility. At this point in the output method, after all the queries have been run, a text file is delivered to the NLP to be cut and pasted into the NLP box.

In the IBM Watson Health software system this step of cutting and pasting the text field into the NLP is required to be accomplished by manual intervention. A proprietary software fix for this operation given the parameters of the platform, is as follows;

The AE 1 provides the cut and paste manual solution as automatic software process, through the use one of the API calls, “stored patient API”, which accepts a plain text string within quotation marks, and basically reads the text file output of the extractor text file from the doctor notes, which the NLP now reads and puts it in as text in the API call of the CDS. The extractor function automatically inserts quotation marks around the text files to enable auto cut and paste into the Attributes. Some further processing of the text file may be required to satisfy the formatting requirements of the API call.

Ideally, the Extractor will be able to output a file which when digested by the CDS's (WfO) NLP permits the generation of a full Treatment Plan. This may not always be the case. In some instances, the text files may require further processing such as in the case of doctors' notes which contain short hand, abbreviations, acronyms, and non-standard idioms. These text files will be further processed by the AE2 termed the translator application. In the end, it is the job of the Extractor to pull from the target system as much required data as possible and organize that data to maximize the effectiveness of the NLP.

The AE 2 operation further supports the EMR entry clinician role of locating missing data, through a further embodiment the AE2. The AE 2 runs a translation operation on the doctor's patient case histories for optimizing the patient's EMR data point recognition for the NLP's capture of Attributes data points. These data points are the key patient metabolic metrics and doctor notes describing the patient's condition, which are required to be entered into the CDS fields. This process entails processing a large number of each doctor User patient cases for translating, correcting and standardizing his language use to address his unique method for patient note taking. This process of standardizing the doctor's patient's EMR case files, is based on an analysis of the patient's doctor's unstructured note taking shorthand and personal use of language which poses barriers to the NLP data capture. This barrier results as a consequence of the doctor's shorthand for abbreviations, acronyms, non-standard drug references and measurements, and a myriad of typos and spelling errors. This standardization process is a onetime process and it's results are recorded and logged in the Hospitals doctor's cloud template for reference every time that specific doctor's has a patient whose EMR is being entered into the Attributes.

The proprietary software automatically analyzes and compares the note taking habits of the doctor for each type of cancer he treats, and creates a customized translation glossary of the doctors' terms and errors for standardization against the NLP's interpretation of the recognized language utilized by the CDS. In this manner, after the Extractor has supplied the text file to the NLP, and populates the Attributes field run, then the Translator will create a comparison file which standardizes the translation of the doctors personal shorthand of unstructured notes for optimal NLP capture of patient data fields.

The key point of the AE 2 translator is to take the doc notes, and convert it into another text file specifically designed to be digested by the NLP.

In a further embodiment the AE 2 translator is an application, which processes a text file such as a doctor's note and replaces any short hand notation with the fully expanded phrases. The purpose of this application is to provide the NLP with a file that it can better recognize. When executing the Translator, there are four files of interest.
1. The input file—for example, a doctor's note
2. The library translation file—this file contains common short hands with the corresponding expanded phrases.
3. The input translation file—this file contains short hands with the corresponding expanded phrases specific to the input file. Common short hands need not be included.
4. The output file—the file that is generated with all short hands replaced with the expanded phrases.
   The file format of both the library translator file and the input translator file are identical. In the first column is the short hand delimited by quotes, in the second column is the expanded phrase delimited by quotes. One short hand per row. For example:

“bx” “biopsy”

“LB” “Left Breast”

These files may be created by the users who are running the Translator application and can be updated at any time, or they may be identified by software designed to locate english language anomalies.

The AE 2 creates two files, the library translation file, and the input translation file. The first column is the abbreviation or shorthand of the doctor, in the second column is the phrase that it needs to be expanded to, e.g., breast biopsy's. It takes the input file from the doctor notes, and takes the library translation file which has the list of shorthand, which the input translation files has a list of the ones that are specific to the doctor, which is read first column. There is one library translation file (translation_library.txt) but many input translation files (one for each doctor). Sample names are:

| | |
|---|---|
| input file | patient_1234_input.txt |
| input translation file | dr_jones_translation.txt |
| output file | patient_1234_output.txt |

When processing the input file, the Translator application will search for any short hand appearing in the input translation file. If found, it will be replaced by the corresponding expanded phrase. The process is repeated for the library translation file. The result is written to the output file. Since the input translation file is processed first, it will take precedence in case any short hand appears in both translation files.

Sample Input:

Patient had bx on LB 3/20/2019.

Sample Output using prior translation file:

Patient had biopsy on Left Breast 3/20/2019.

The doctor uses BX as biopsy's, and no one else except that doctor used BX for biopsy's, you have one file for each doctors library, and AE 2 creates an additional access input file for each doctor to auto log in his unique idioms.

Once you have both files then the NLP is optimized to translate all of the doctor notes from his shorthand, and the NLP can then read doctor notes, having translated the terms that it did not recognize into the words that it does.

A manual check and visual search of the doctor's patient's case may be required by the EMR data entry person to verify and address any use of language that remains a ‘mystery’ to the NLP survey. This step precedes all patient Attributes capture of data points for the CDS.

The AE 3 embodiment provides for a predictive additional operation designed to anticipate the locations of missing Attribute data points. This operation is activated either manually by the clinician, or semi-automatically in response to the operations of AE2 and either independently of or in conjunction with, the completion of the first capture and populate pass of the NLP, on the patient's EMR for the Attributes form.

Under AE 1 operations, after the first NLP automatic capture, missing data is indicated by the CDS, which is then the basis for the next round of Attributes questions. The clinician then manually searches for and fills in the missing data. Then another round of fields are presented by the CDS based on the previous answers. The NLP runs the fill ins and then the clinician has to again find the missing date. The clinician locates the missing data, each time requiring a manual search, and this process continues until all of the required fields have been completed.

There are a number of reasons why WfO might report that an attribute is missing data: the data may not have been available in the target system; the AE 1 extractor application may not have extracted it correctly or the NLP may not have been able to infer the Attribute value. While this issue may be corrected in certain circumstances, it cannot be eliminated. In practice, the missing item would be tracked down. When a user has EMR access and the attribute exists in the EMR, then it could be found by searching the appropriate location. However, it is possible that the patient's nurse or doctor would have to be contacted. A one-time contact is inconvenient, the real problem occurs when the missing attribute value is acquired and entering it triggers successive missing attributes. In cancer, there are many instances where the required attributes cannot be known ahead of time. It is due to this property of the disease that these cascading missing attributes can appear. The purpose of the AE 3 predictor application is to minimize these follow-up contacts, by predicting in advance, from learned experience, which patient missing data points will be required.

Initially, this application is designed for user input. This is due to the fact that the CDS (WfO) API does not include a call to query for missing attributes. This methodology provides for a workaround to address that issue for a semi-automatic result. If that call is implemented by the CDS, it will then be possible, with this methodology, to fully automate this application. The application will function as follows:
1. Upon entering patient data into CDS (WfO), a missing attribute value is reported.
2. POP user opens the Predictor application and enters the cancer type.
3. The missing attribute is selected from the dropdown.
4. If there are no entries in the cascading attribute list, the missing attribute value is acquired and entered.
5. If no more missing attribute values are reported by the CDS (WfO), exit.
6. If one or more new missing attributes are reported by the CDS, select default “All” in the value dropdown.
7. In the cascading attribute list, select each new missing attribute and Save.
   In a future patient case:
1. Upon entering patient data into WfO, the same missing attribute value as above is reported.
2. View the cascading attribute list and acquire each attribute listed.
   After entering many missing attributes in the AE 3 predictor operation and AE 2 translator operation, a detailed list of cascading missing attributes will be compiled. This list can be further refined by entering specific values in the value dropdown. With this information, the number of follow-up queries can be significantly reduced.

Because this process is replicated for every patient's Attributes CDS entry, as Attribute entries accumulate, we can predict when ever the attributes ask for a specific missing field, we know from the previous experience and search, based on AE 3 and the application of machine learning, what the next likely Attribute request will be, and we look for it, and ask for it in advance, because now we know that given the answer that we have just located and given, that we will now need the next four fields, which we can anticipate, locate and populate accordingly.

Because we are using the WfO IBM Attributes form, which is based on a given set of answers, which is the basis for triggering the next one, our software machine learns the pattern of the next needed fields and then our API learns from the fields requested and we then automatically ask for the next fields because we know that Watson is going to need the next three, we ask for them in advance, once we know how Watson works for each cancer.

Then the next time the NLP shows that an EMR field is missing, and it can now enter the data for the field for the Attributes from a dropdown menu in our POP program, or anticipated next fields, and then because it has been entered previously, the program knows where it is. In this methodology the AE 3 provides utilization of the Watson repeated experience, to avoid having to go back to the EMR or doctor more then once to pick up the missing attributes and thereby solves a major time consuming issue of multiple back and forth requests from the Doctor's service to locate missing information.

This will require a combination of machine learning software based on patterning and initial human operators to provide the manual correction and learning. This can be done eventually with the aggregation of data for this process either from machine learning utilizing the aggregation and collection of large numbers of EMR files, or from standard database search and organizational recording.

As more and more hospital patient EMR file data points are entered into the CDS, the system self learns and transitions from a manual to a semi-automatic methodology into a fully automated process. The methodology produces a unique and custom designed roadmap for each specific hospital and doctor, for optimizing the CDS capacity to seamlessly capture patient EMR data, and making it agnostic and available to any artificial intelligence CDS platform, with the proper permissions, thereby solving a major bottleneck issue plaguing all Hospital EHR and EMR systems.

The value of this algorithm is to consolidate the requirement for further information to one set of data. Watson for Oncology requests further information from the GUI in a serial format and in a practical sense requires multiple manual entry integrations on the EMR data, which is time consuming and disruptive for the nursing staff. The ability to predict the data required in a comprehensive approach, is key for the day to day integration into hospital routines. This inability to seamlessly manage the patient EMR data points, is a major shortfall in the adoption of any CDS system and specifically for the IBM Watson for Oncology application, which this methodology's approach will address.

The AE 4 addresses programmed communication between external entities such as aggregating multiple Hospital's EMR data, Biometric medical wearable's, 3^{rd} party Applications, lab results, the Internet Websites, external Cloud access and the portal application.

To illustrate, consider a case where a patient is being monitored in real time using a remote biometric sensor 42 for a health condition, e.g. a cardiac monitor. Here the AE 4 is responsible for managing the real time device signal and transmitting it to the RTC 20. Machine learning 28 and other algorithms execute within the RTC 20 to capture and analyze the input monitor signal. If the real time analysis detects an anomaly in the monitor signal, the RTC 20 triggers an alert, which is posted to the physician dashboard 14. The alert is a multi-part message which contains information specific to the event, e.g. cardiac monitoring, for the patient, along with a priority estimate supplied by an analysis of the ML 28. The automatic alert is simultaneously communicated to the AutoE 36, which uses internal logic to assess whether automation settings are impacted by the alert. In an example instance, the AutoE 36 may determine that the alert is max priority, which requires adjustment of settings for the CDS 32 for auto-run mode.

A further embodiment of the AE 4 pathway is designed to augment and support the integration of data captured from an aggregation of Portal hospitals under membership for the purpose of facilitating research validating the protocol treatment recommendations with the patient Outcomes. This data is key to developing refinements to the treatment protocols based on real time outcomes, as demonstrated by the ongoing collection of data.

As regards the AE 4, its functionality is to also aggregate, anonymously, patient data as the disease progresses over extended time frames. When the Patient returns to the Portal, over the years, hopefully through successful remissions, as the disease becomes a chronic condition under maintenance, the patient's use of the Portal captures key data valuable to future Diagnostics.

AE 4 is also an R&D tool designed to support the aggregation and pre-processing of vast amounts of CDS corpus insurance case histories, hospital anonymous patient outcomes, and for the correlation of protocol regimen treatments with outcomes, both macro long term, and micro as in outcomes related to on-going Chemo therapy cycles.

Because patient's often do not return to the same medical facility from which they last received treatment, the ongoing tracking of patient outcomes is impossible. From treatment to remission, his use of the Portal methodology becomes a key constant and a source of on his going data collection regardless of which doctor of hospital he is using. This information is capture anonymously and organized via the AE 4 to provide guidance for future patient treatment recommendations based on previously correlated treatments with outcomes.

In summary, the AutoE 36 controls the temporal performance of the portal platform 10 and enables several modes of operation: asynchronous, scheduled, on-demand and real time. The AutoE 36 works in conjunction with the RTC 20 in monitoring and responding to system events to control the operations of different system modules, e.g. the AUI 12, DVE 30 and CDS 32.

Further, the AutoE 36 works in conjunction with the machine learning module 28 in the RTC 20, applying optimization techniques to reduce the potential for alert-fatigue, a major factor that has plagued EHR systems and is associated with clinician burnout and diminished workflow performance. The AutoE 36 is designed as a flexible module with input and output connecters enabling it to tie together and thereby regulate different system pathways, e.g. controlling the CDS 32 with its input from the DVE 30 and output back to the RTC 20, another path controlling regulation of interaction between the DVE 30 and the EHR 38 and a third path indicating programmed communication between external HIE Network 40 entities and the portal 10. The extensibility of the Portal Methodology is a novel and unique feature of the architecture design which is the basis for the flexibility of the Portal to adapt to future A.I. developments and customizations.

D. Data Validation Engine (DVE) 30

The DVE 30 constitutes an ETL (extract, transform, load) engine. The DVE's 30 primary function is to serve as patient EMR data input to the CDS (this input record for WfO is termed the ‘Attributes Form’ (AF)). The DVE 30 has the critical task of helping to automate the process of preparing the input CDS data—a process that currently often involves manual data entry—through a multi-step methodology.

In a typical scenario, some fields required for the CDS 32 input will already have been recorded in the patient EMR 38 and transferred to the CDS AF, through the operations of the AutoE 36 and the AE 1, AE 2, and AE 3 functions. Other required fields will have to be located by the EMR nurse or data entry technician and manually loaded into the AF. The DVE 30 is designed to facilitate a process in which these manual steps can be automated and thereby eliminated, leading towards fully automated workflow for this data transfer, which can recorded to the hospital cloud

In the first step, a data map will be constructed with address links to the location of each EMR data component required by the CDS 32. As noted, some data may already exist within the EMR 38, other data will be located by the technician and that location recorded. Note also that these data components will typically consist of a mixture of structured and unstructured data.

The second step will be to perform data extraction and normalization, via AutoE 36, i.e. to ensure that data for each (structured) element is in the appropriate format, and the values within proper range, for the input Attributes Form. All normalized data will be checked for compliance with the HL7 FHIR STU 3 standard.

In the third step, cognitive analysis employing Natural Language Processing 22 is used to analyze unstructured data and, where possible, is transformed into structured (e.g. numeric valued data that can be stored in tables or databases) equivalents. NLP 22 processing of unstructured data (e.g. doctor free text or dictated notes, video, audio transcripts, medical images) is context-aware so that alerts or alarms can be automatically triggered by NLP 22 analysis.

Consider, for example, an instance of Pathology Lab reports describing tumor growth for an oncology patient, entered as free text and digitally scanned as unstructured data. NLP 22 will intelligently read the digitized notes, record the relevant tumor size data and check the information against earlier recorded data for this patient, as well as pertinent clinical status indicators. The result could be an alert or alarm automatically added to the physician Dashboard 14. Simultaneously, the new data can activate the real time controller to trigger the AutoE 36 to perform a re-run of the CDS 32 diagnostics and generate a new report for the clinical staff.

In the final step, any remaining errors or discrepancies in the input AF are flagged with alerts posted to the dashboard 14 enabling technicians to take further action for resolution of these data elements. The processed AF is stored in portal database 34 and available on demand for CDS 32 analysis. Stored patient AF data is searchable for privileged users to access patient history. In this final step, the DVE 30 will generate a report compiling a list of all such potentially significant clinical data. This report will be stored in the portal database 34 and accessible to the physician on the user dashboard 14. The report may also be transmitted to hospital personnel to alert them to the data issues and to assist them with optimizing CDS 32 workflow.

The CDS 32 entry process described above is designed to minimize manual data entry steps and other data roadblocks to efficient CDS 32 operation. Over time, this case by case customization process promotes the transition from semi-automatic to fully automated CDS 32 operation. The data maps and reports as described in the above process will be stored in cloud portal database 34 as a template unique to the particular healthcare organization. These templates may be shared through the HIE Network 40 to assist other healthcare organizations in their data automation efforts and promote interoperability.

In a further aspect of the present invention, the portal platform 10 will extract patient EHR 38 data in a format compliant to the emerging HL7 FHIR STU 3 standard, thereby enhancing EHR interoperability by promoting adoption of this FHIR standard.

E. Clinical Decision Support (CDS)—IBM Watson for Oncology 32

The CDS system 32 (e.g., IBM WfO in our preferred embodiment) is an important core component in the inventive portal 10. The CDS 32 is a distinct executable application, and is preferably cloud-based. The inventive portal architecture 10 envisions that the application can interact with the CDS 32 either directly in an on-demand mode, through a scheduler or asynchronously in real time, under control of the RTC 20 and the AutoE 30.

CDS systems 32, e.g. IBM WfO, perform advanced cognitive analysis of the patient's clinical data, considering many evidence sources such as established clinical guidelines, medical literature, clinical trials data and other patient's records, to provide diagnostic and treatment advice and recommendations to support the clinician's decision-making in the patient's care journey. WfO, for example, draws on 300 medical journals, more than 200 textbooks, and nearly 15 million pages of text in its digital ‘corpus’ to support its cognitive analysis. CDS systems 32 require the patient clinical record for input, the Attributes Form for WfO, and consists of selected data from the patient EMR 38 sufficient to fully characterize the current oncological status of the patient. The output from running the CDS 32 analysis comprises a patient diagnostic report for the clinician that presents a hierarchically ordered list of evidence-based recommendations for diagnosis and treatment.

Physicians can easily view the evidence underlying each recommendation by clicking on links to the medical literature, clinical trials data and other information that was used by the AI system in producing that specific result.

CDS 32 data, both input and output, will be time stamped and archived in the portal database 34, providing a curated longitudinal health record for the patient.

F. 3d Party Apps 42, Remote Biometric Sensors 44

The inventive portal is designed for extensibility and scalability; the use of open protocol software standards such as FHIR and SMART enables third-party applications to connect to the invention platform. Further, API's will be available comprehensively incorporating portal platform 10 functionality that enable third-party developers to develop custom applications to run on top of the inventive portal 10. Flourishing ecosystems of third-party apps are already developing around major EHR's such as Cerner and Epic and the portal system will support integration of these as well as newly deployed apps. These apps cover a broad spectrum of medical applications such as: support for specific medical conditions, e.g. cardiovascular or diabetes, apps related to personal health and activity data, apps to support consumers' sharing of medical data, personal medication management.

Patient generated health data (PGHD) from IoT (Internet of Things) devices such as wearables and biometric sensors 42 is a trend that is growing substantially and a major factor in the evolution towards real time health systems. These devices generate a wide range of health data and include fitness and activity trackers, blood pressure and blood glucose readings, electrocardiograms and digital asthma inhalers. The portal 10 will include interfaces built on open healthcare protocols to enable connection to biometric sensor devices 42 and extraction of their data in real time. Machine learning algorithms 28 will be applied to analyze incoming signals for detection of anomalies or significant events, using the RTC 20 and AutoE 36 to trigger alerts and alarms when required.

G. Portal Database 34, EHR/EMR 38, HIE Network 40

The Portal Database 34 and EHR/EMR 38 are preferably cloud-based and may be built using cloud platforms such as Amazon Web Services (AWS), Azure (Microsoft), Google Cloud.

Advancements in the efficacy of CDS 32 systems and healthcare analytics more generally are dependent on availability of large clinical datasets that represent a spectrum of disease types, demographics, diagnostics, treatment regimens and outcomes. In recent years a number of large scale initiatives have begun to address the need for aggregated datasets including initiatives from National Cancer Institute (NCI) and the American Society of Clinical Oncology (ASCO) CancerLinq. Datasets that enable analysis of patients' longitudinal health records from initial diagnosis through treatment to outcome and also to follow-on remission history will be particularly valuable.

The present portal platform 10 design has embodiments for promoting oncology data aggregation; a key aspect is the data standardization and normalization in the patient EMR 38 that results from the portal DVE 30 processing prior to input to the CDS 32. This process ensures optimally curated data, i.e. an inherent data uniformity for all such clinical data that is transferred to cloud storage that minimizes or eliminates the necessity of burdensome and time-consuming additional data cleaning operation tasks that are commonly required in clinical databases.

In a further embodiment the tools provided by the invention portal platform 10 facilitate continuous longitudinal tracking and recording of the patient health record through all stages of the healthcare journey. This patient-specific data will be aggregated over time as extracted from multiple runs of the CDS 32 that include the EMR as embodied in the CDS input Attributes Form as well as the diagnostic and treatment recommendation results output from the CDS 32. Additionally, the cloud aggregated patient data can include information input directly by the patient including unstructured notes, processed with NLP 22, describing symptoms, lifestyle changes and other pertinent data.

In an exemplary embodiment data aggregation promoted by the invention portal platform 10, evolves over time to represent multiple hospitals and a large patient cohort; this process creates an important resource to be shared widely with the oncology healthcare community, providing new insights and approaches for all stakeholders.

Experts are heralding the arrival of the new era of ‘big data’ in healthcare analytics and the use of Clinical Decision Support Systems (CDS) are widely recognized as a crucial driver in what is expected to be a fundamental transformation of the healthcare system towards evidence-based care, value-based payment models and quality metrics. Progress in this direction, however, is still slowed by the continuing problems in lack of EHR capabilities for integration and interoperability.

A further embodiment to assist in overcoming these problems, the invention platform features from the ground-up design with technology designed to facilitate integration within the EHR as well as interoperability between different EHR systems. Specifically, internal EHR integration is promoted by the use of open standards such as FHIR and SMART.

To ensure that the portal platform 10 will be interoperable between diverse EHR systems, the portal database 34 may be used for storage of relevant resource data specific to each particular EHR. For example, Cerner resources describing e.g. patient demographics, encounters, medications, may have differences from a rival EHR, e.g. Epic counterparts. The portal 10 allows these EHR-specific data elements to be applied as required based on the assigned EHR deployment.

2. Embodiment Diagrams

A. AUI Alarms for Patient Diary Entries of Contraindicated Agents

The embodiment diagrammed in FIG. 2 demonstrates an embodiment of the inventive real-time, intelligent operations of the portal system 10 in generating physician alerts of possible contraindicated agents consumed by the patient.

In Step 62 the doctor logs into the AUI 12, accessing the physician dashboard 14. A new chemotherapy order set is entered in Step 64, causing the patient EMR to be updated in the portal database 34, Step 66. Following confirmation of this transaction, Step 68, the doctor checks the dashboard 14 for possible alerts for this patient.

The patient logs in at Step 76 and accesses the patient dashboard 14. In Step 80 the patient records a diary entry, indicating in his text items, a list of his daily food intake, a list of the nutritional supplements he is taking, and any other medications, prescribed or over the counter, and the contents he had at a recent meal (shellfish). NLP 22 in the RTC 20 auto-analyzes the diary entry; one of the NLP 22 stored ‘contexts’ is ‘food items’ which leads it to extract a list of those items, Steps 82, 84. The newly entered food items are then automatically analyzed using a machine learning algorithm 28 executing in the RTC 20 which tests for similarity/compatibility with the stored patient profile, Step 86. If this test is negative, no further actions result.

If the ML 28 comparison test indicates a possible significant anomaly in Step 88 for the ‘shellfish’ food item, a further ML 28 automated analysis results at Step 90 with a scan of contraindicated agents as listed in the stored Guidelines associated with the recently submitted chemo orderset. If the scan identifies ‘shellfish’ as a contraindicated food substance, an alert is automatically generated and forwarded to the doctor Dashboard 14, Step 94. If the scan test returns negative, a further analysis will be invoked using the system 10, FIG. 1, Predictive Analytics 26 (PA) module, Step 72. The PA 26 tool allows queries to be run against large cohorts of patients with similar profiles, Step 74, which may uncover results that differ from the more localized analysis performed in Steps 86, 88.

If the PA 26 results are negative, no further action results. If the results are positive, an alert is generated and sent to the physician at Step 94.

When the physician receives an alert, it is evaluated, Step 98, for follow-on action. This action may, for example, involve ordering new labs, Step 100, and forwarding notifications to the patient, Step 102.

B. AUI Alarms from Patient Biometric Sensors

The embodiment diagrammed in FIG. 3 demonstrates the real-time, intelligent operations of the portal system 10 in generating physician alerts based on monitoring of signals generated by sensors that may be incorporated in patient biometric sensor devices 42, such as cardiac monitors.

In Step 112 the doctor logs into the system, accessing the physician dashboard 14. A new chemotherapy order set is entered in Step 114. The doctor reviews Guidelines for possible adverse effects and risk factors for the patient with the new treatment regimen, taking into account the patient's history of cardiovascular (CV) issues which includes arrhythmia, steps 116 and 118. Doctor notes that arrhythmia is a known risk factor with the new regimen.

In step 120 the doctor orders a biometric sensor device 42 for the patient to monitor cardiac signals in real-time. The patient is sent a notification to his dashboard 14 in step 122 and his EMR is updated in the portal database 34 in step 124. These updates are confirmed in step 126.

In step 132 the patient activates the biometric sensor device 42 which is then registered on the portal platform 10, step 134. Incoming real-time signals from the biometric sensor device 42 are received, sampled and analyzed with machine learning algorithms 28 that execute on the portal platform 10 in step 136. These ML algorithms 28 have been trained to detect anomalies in the cardiac signal indicative of arrhythmia, step 138.

At some time the patient may suffer an adverse reaction to the new chemo regimen, step 144. Real-time monitoring and ML analysis 28 detects the event, step 144, which triggers an alert that is posted to the clinician dashboard 14 in step 146. The ML algorithm 28 assesses the severity of the detected event and assigns a priority level to the triggered alert in step 148. If no adverse effect, default to continue monitoring.

In step 128, the physician observes an alert that has been posted to his dashboard 14. If the alert indicates a low to moderate priority level for the detected CV event, he has the option to re-run the CDS 32 and generate a new diagnostic report for the patient, step 130.

If the alert indicates a high priority for the detected CV event, it triggers an auto-run of the CDS 32 in step 150, whereby the CDS 32 input Attributes Form is updated with current information from the Doctor's Dashboard, step 152. The CDS 32 run results in generation of a new diagnostic report which is posted to the clinician dashboard 14 in step 154. The patient subsequently receives notification that updates are available and to contact his physician, step 156.

C. NLP EMR Data Entry Processing from EHR

The embodiment diagrammed in FIG. 4 demonstrates an embodiment of the inventive real-time, intelligent operations of the portal system 10 in ingesting data from the EMR which resides in the EHR 38 into the a Attributes Form which is required input for the CDS 32 to run the Patient Diagnostic Report (PDR). This inventive process is designed to transition the workflow from manual to semi-automated and ultimately to fully automated performance.

In Step 202 the EMR technician logs into the system, accessing the technician dashboard 14. In step 204 the tech enters the patient ID to access patient records from the EHR 38. The tech objective is to fully populate the input CDS 32 Attributes Form with the required, up-to-date patient EMR data, step 206. Prior to this step the Auto Engine 1 has run a manual assisted semi automatic ‘standardization’ of the doctor's unstructured note taking shorthand and corrected and optimized all language anomalies to provide maximum NLP capture of patient EMR for the Attributes CDS run. The first step in this process is to initiate an auto-scan of EHR 38 to retrieve EMR data that resides in the EHR, 38 step 208. Invalid data may be identified by Attributes Form auto validation and added to auto scan report. The auto-scan generates a visual map display indicating the status of each required data element, step 210. This visual display may be color-coded to indicate the status of each data component: data exists and is valid, data is missing, or data exists but is invalid, step 210, 212.

In step 212, the EMR tech has completed evaluation of the EHR 38 auto-scan and has determined whether there are unresolved data issues. If there are none, data entry into the CDS 32 Attributes Form is completed and a confirmation notification sent to the EMR tech dashboard 14 in Step 214. No alerts are generated and the tech continues in step 216.

If the EHR 38 auto-scan has revealed data issues, an alert is sent to the EMR tech dashboard 14, along with an electronic copy of the Data Entry Status report in step 230. In step 234 the EMR tech retrieves the portal database 34 stored Attributes Form Template which contains pertinent metadata that shows, for each required EMR data element, the electronic address of the data element in the EHR 38, the required value bounds on the data element and its normalization requirements (eg. units of measurement).

In step 236 the EMR tech commences to evaluate and fix the data issues element by element, starting with a determination of whether the data element is missing. If the data is missing, the tech examines the Attributes Template to see whether an electronic link address is available, step 238. If a link is available, the missing data is retrieved in step 240. Then in step 242 the retrieved data is examined to see whether it is in unstructured format (e.g. handwritten notes, medical images, audio) or in structured format. If the data is unstructured, NLP 22 is applied in step 244 to parse the data into a structured form; subsequently, the Attributes Form is auto-populated with this data element in step 246.

If a link address is unavailable for the missing data element in step 238 the EMR tech will manually locate the data in step 218. Once located, the data is evaluated in terms of its format, unstructured or structured in step 220. If unstructured, it will be parsed as before in step 244. In step 222 the tech updates the Attributes Form Template with the newly discovered electronic address for this missing data element.

Once it is determined that the data elements exists, that element must be determined to be valid in Step 224. In step 226 the data element is inspected for normalization and if required it is auto-normalized, step 228 prior to advancing to step 248 wherein a further test checks to see whether the data value satisfies the boundary requirements as recorded for this element in the Attributes Form Template. If the data is outside required bounds, the EMR tech follows up with the hospital EMR coordinator, step 250, who is automatically notified of the existence of EMR data issues. If the data value is within required bounds, processing of this data element is complete and the EMR tech advances to the next data element at step 204.

The result of the initial test of the data element extracted by the EMR scan may show that data is present, however, that data still must be tested for validity, step 224, 226, 228, 248 and 250.

The hospital EMR Coordinator receives the Data Entry Status report in step 250. EMR coordinator resolves invalid data issues, The EMR tech receives notification from the hospital EMR coordinator that data issues related to this patient's EMR have been resolved along with a report detailing steps that were implemented to achieve resolution in step 252. EMR tech initiates CDS patient diagnostic run. Step 254.

D. CDS and Predictive Analytics

The embodiment diagrammed in FIG. 5 demonstrates an embodiment of the inventive real-time, intelligent operations of the portal system 10 in linking the CDS 32 with the Predictive Analytics module 26 to extract additional clinical insights to advance the standard of care in the healthcare organization.

The doctor has ordered a CDS 32 run for his cancer patient, for whom he has recently ordered a new chemotherapy regimen, step 302. The Doctor inspects the current CDS 32 system timing settings in the Automation Engine 36 to determine whether there are pre-settings for scheduled or auto-run mode. The CDS runs and returns a new diagnostic report to the doctor dashboard with a hierarchical list of diagnostic and treatment recommendations for the patient, the doctor reviews the new report in step 304.

In Step 306 the physician logs into the system, accessing the physician dashboard 14.

The doctor next retrieves the cancer patient's EMR from portal database 34 storage.

The physician is interested in obtaining additional clinical information pertinent to the patient's case and to the newly received CDS 32 report. For this purpose the doctor accesses the PA module 26 and selects from a list of possible target databases for his queries, step 312.

The physician compiles a list of patient-specific queries to be submitted to the PA system step 314:
mortality risk
probability of hospital readmission
risk of toxicity for new chemo regimen
contraindicated agents
additional characterization of tumor
applicability of genomics therapies
   These queries are submitted and executed on the PA system 26, which auto-compiles a report with extracted information in step 316.

The PA 26 report is combined with the new CDS 32 report, step 316 and the combined report circulated by the doctor to colleagues for discussion at the next hospital Tumor Board meeting, step 318.

The physician, having reviewed the information returned by the PA 26 system, decides to expedite data retrieval by setting automatic settings in the Automation Engine 36 on the PA 26 system; these settings link the PA 26 system and the CDS 32 system and enable a pre-stored list of queries to autorun on the PA 26 system, step 320.

A new CDS 32 run is initiated by the doctor in step 322. In step 322 and 324 the CDS completes and forwards a new diagnostic/therapeutic recommendations list to the doctor dashboard 14. The PA 26 settings in the Automation Engine 36 triggers the PA 26 auto-run with the pre-stored queries related to the patient case, step 328. The PA 26 results are auto-compiled into the new report in step 330.

The new CDS 32 and PA 26 results appear on the doctor dashboard 14 in step 332.

The doctor reviews the new results, sends notification to patient to schedule consult in step 334.

E. AUI—User Profile with Machine Learning

The embodiment diagrammed in FIG. 6 demonstrates an embodiment of the inventive real-time, intelligent operations of the portal system 10 Adaptive User Interface 12 which uses machine learning 28 in optimization of the user profile to enhance user engagement.

In step 352 the cancer patient logs into the AUI 12/dashboard 14. The patient has recently seen his cardiologist and received a new prescription for his existing hypertension condition, step 354. The patient enters new medication information into the Diary on the portal dashboard 14, step 356. Using NLP 22, the portal system 10 automatically translates the unstructured notes data into structured format in step 358. In step 360 an ML algorithm 28 automatically runs on the updated patient profile and detects a significant change due to new medication information.

The change in patient profile triggers an alert which posts to the doctor dashboard 14 in step 362.

In step 364 the physician logs into the AUI 12/dashboard 14. In step 366 the physician observes the alert for his cancer patient that indicates a change in the patient profile. Information posted with the alert indicates a change in the patient CV state which is known to the doctor but had previously been considered minor.

In step 368 the physician considers that, despite lack of warnings from recent CDS 32 runs for the patient, that there may be a connection between patient's changed CV status and the new chemo regimen recently prescribed by the doctor.

In step 370 the doctor determines to conduct further investigation leveraging the PA system 26 that is linked to the CDS 32 and to external databases such as ASCO CancerLinQ 40.

In step 372 the PA system is used to analyze data from a large cohort of patients with similar cancer diagnosis, treatment regimen, and corresponding CV condition, examining data from recent medical research as well. The PA system 26 generates predictive results indicating a small but significant risk of increased hypertension associated with the new chemo regimen, step 372. This finding is consistent with the newly submitted information from the patient regarding his medication changes.

Based on the PA 26 findings, the doctor orders a re-run of the CDS 32 and examines new options presented in the CDS 32 report, step 374.

As a precaution, the doctor adjusts settings for auto-run using the Automation Engine 36 on both the CDS 32 and PA 26 systems to trigger automatically if new data input from the patient indicates any further changes in CV status, step 376.

Based on the new CDS 32 and PA 26 data, the doctor notifies the patient to schedule a consultation, step 378.

The patient, in step 380, upon logging in again to the AUI 12/dashboard 14, notices changes on his user interface in the form of new content options presented to him, e.g. news on cardiac health and lifestyle, upcoming seminars and events and an invitation to join support groups for patients with cancer and CV conditions.

F. Use of CDS/WFO to Create Longitudinal Health Record on Blockchain

The embodiment diagrammed in FIG. 7 demonstrates an embodiment of the inventive real-time, intelligent operations of the portal system 10 wherein a longitudinal health record is created for the patient, recorded on a Health Information Exchange Network blockchain 40 and shared among all stakeholders in the patient's care circle.

In step 402 the physician logs into the AUI 12/dashboard 14 and in step 404 accesses his cancer patient's EMR which is stored in the-EHR Hospital database 34. The doctor orders a CDS/WfO 32 run for the patient, step 406 and receives a new CDS patient diagnostic report 18 on the AUI 12/dashboard 14, step 408. Physician reviews the new report and schedules a consult with patient, step 410.

The doctor is participating in a pilot test of a HIE blockchain-based 40 system which provides direct, near-real time access to patients' longitudinal CDS/WfO 32 health records for clinical and financial stakeholders, step 412.

Doctor clicks on ‘HIE Access’ button on dashboard 14 to launch an app that connects to the HIE HIPAA secured blockchain 40 pilot program, step 414.

Using the app, doctor investigates to confirm that patient has granted permissions for submitting patient CDS/WfO 32 data to the pilot program, step 416. If not confirmed, doctor exits app in step 418 and records not to ask patient's permission at next consult.

If permissions are confirmed, the doctor requests submission of current CDS 32 data comprised of the Attributes input form along with the CDS Patient Diagnostic Report 18 containing clinical diagnostic and treatment recommendations, step 420. The app automatically processes the transaction on the HIE blockchain 40 and returns a confirmation, step 422. The updated health record for the patient is available in near-real time to all permissioned users on the blockchain, step 424.

The patient has given permission for his HIE blockchain 40 data to be viewable for research purposes using his public (non-identifiable) key, step 426.

In step 428, notifications of the patient's record update have automatically been distributed to all participating stakeholders in the HIE blockchain 40. One such participant is a cancer researcher who is interested in cases that share clinical profiles similar to the patient's. The researcher has used the HIE app to configure settings to automatically be notified when new data matching her interests appears on the blockchain 40, step 430.

In step 432, a notification is received by the researcher who logs into the HIE app and is able to view and study the patient's de-identified clinical record that has just been updated.

The patient's family doctor receives in near-real time a notification of an update to the patient record, step 434. Using the HIE app, the family doctor checks to see whether she has received permissions, including the private blockchain key to unlock the patient's newly updated record on the HIE blockchain 40. If not, the doctor makes a note to contact the patient in step 438. If yes, the family doctor logs into HIE app and, using the private key provided by the patient, reviews the updated health record containing the latest CDS/WfO results step 440. The family doctor notifies the patient by email that she has reviewed the updates, step 442.

In step 444, the hospital billing office receives near-real time electronic notification of update to patient's health record. In step 446 the billing office staff uses the HIE app to access the patient's health record on the HIE blockchain 40. Billing office is permissioned with the patient's private key to view the identified record on the blockchain 40, which indicates that a run of CDS/WfO 32 has just occurred, step 448. In step 450 the billing office notes this information, looks up the CDS/WfO 32 contract with the hospital and adjust invoice and financial records accordingly.

While there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A real time intelligent healthcare information system operable with a clinical decision support (CDS) system, comprising:
a. An adaptive user interface (AUI) module for receiving from a medical professional medical guidelines for the patient upon authentication of credentials of the medical professional and for receiving health-related information from a patient upon authentication of credentials of the patient, and for outputting clinical recommendations to the authenticated medical professional and patient, the AUI module including:
i. A dashboard to display clinical recommendations from the CDS system to one of medical professional and patient upon request;
ii. A voice assistant responsive to voice commands of one of authenticated medical professional and patient, the voice assistant configured to one of transcribe speech into text and text into speech; and
iii. A clinical decision output module for processing clinical recommendations from the CDS system and outputting the processed clinical recommendations to one of the dashboard and voice assistant;
b. A real time controller for processing data between (i) the medical professional, patient, a biometric sensor monitoring the patient, and electronic health records of the patient; and (ii) the CDS system, the real time controller including:
i. A natural language processor for understanding text received from the AUI and in unstructured data in medical reports of the electronic health records;
ii. A corpus query module responsive to the medical professional for searching the corpus of medical research reports;
iii. A predictive analytics module responsive to the medical professional or patient for analyzing the patient's current and historical patient data including medical research data from the corpus query module, and for providing clinical recommendations to the medical professional or patient based on an analysis of the patient's current and historical medical data and medical research data from the corpus query module; and
iv. A machine learning module for analyzing patterns in the current and historical patient data and medical research data including identifying anomalies in real time input signals from the remote biometric sensor monitoring the patient;
c. A data validation engine (DVE) for interfacing with the real time controller and the CDS system, the DVE configured to one of verifying compliance of unstructured patient data and conforming non-compliant unstructured patient data prior to input to the CDS system; and
d. An automation engine for interfacing with (i) the real time controller and (ii) one of the patient's biometric sensor and electronic health records, the automation engine transmitting data from said one of the patient's biometric sensor and electronic health records to the real time controller when triggered by notification of availability of updates from said one of the patient's biometric sensor and electronic health records.

2. A method for providing real time healthcare information using a clinical decision support (CDS) system, comprising the steps of:
a. Using an adaptive user interface (AUI) module for receiving from a medical professional medical guidelines for the patient upon authentication of credentials of the medical professional and for receiving health-related information from a patient upon authentication of credentials of the patient, and for outputting clinical recommendations to the authenticated medical professional and patient, the AUI module including:
i. A dashboard to display at least one of clinical recommendations from the CDS system to one of medical professional and patient upon request;
ii. A voice assistant responsive to voice commands of one of authenticated medical professional and patient, the voice assistant configured to one of transcribe speech into text and text into speech; and
iii. A clinical decision output module for processing clinical recommendations from the CDS system and outputting the processed clinical recommendations to one of the dashboard and voice assistant;
b. Using a real time controller for processing data between (i) the medical professional, patient, a biometric sensor monitoring the patient, and electronic health records of the patient; and (ii) the CDS system, the real time controller including:
i. A natural language processor for understanding text received from the AUI and in unstructured data in medical reports of the electronic health records;
ii. A corpus query module responsive to the medical professional for searching the corpus of medical research reports;
iii. A predictive analytics module responsive to the medical professional or patient for analyzing the patient's current and historical patient data including medical research data from the corpus query module, and for providing clinical recommendations to the medical professional or patient based on an analysis of the patient's current and historical medical data and medical research data from the corpus query module; and
iv. A machine learning module for analyzing patterns in the current and historical patient data and medical research data including identifying anomalies in real time input signals from the remote biometric sensor monitoring the patient;
c. Using a data validation engine (DVE) for interfacing with the real time controller and the CDS system, the DVE configured to one of verifying compliance of unstructured patient data and conforming non-compliant unstructured patient data prior to input to the CDS system; and
d. Using an automation engine for interfacing with (i) the real time controller and (ii) one of the patient's biometric sensor and electronic health records, the automation engine transmitting data from said one of the patient's biometric sensor and electronic health records to the real time controller when triggered by notification of availability of updates from said one of the patient's biometric sensor and electronic health records.
